(19) United States
(12) Patent Application Publication
(10) Pub. No.: US 2022/0048056 A1
(43) Pub. Date: Feb. 17, 2022

(54) LIQUID DISPENSER

(71) Applicant: OHHY MEDIA LLC, Ball Ground, GA (US)

(72) Inventors: Mark Terry, Kennesaw, GA (US); David Howard, Marietta, GA (US); Grant Terry, Kennesaw, GA (US); Kyle Terry, Kennesaw, GA (US)

(21) Appl. No.: 17/375,812

(22) Filed: Jul. 14, 2021

Related U.S. Application Data
(60) Provisional application No. 63/066,091, filed on Aug. 14, 2020.

(51) Int. Cl. B05B 11/00; A61L 2/22

(52) U.S. Cl. CPC B05B 11/0056; B05B 11/3002; A61L 2202/182; A61L 2202/16; A61L 2/22

(57) ABSTRACT

A liquid dispenser for containing and dispensing a sanitizing liquid is described that comprises a container body defining a container chamber in fluid communication with both a dispenser port and a fill port. The fluid dispenser comprises an atomizer operably connected to the dispenser port and a cover operably connectable to the container body and movable between a cover-open position in which the fill port is exposed and a cover-closed position in which the fill port is covered.

### RELATED APPLICATION

This application claims the benefit of priority to U.S. Provisional Application No. 63/066,091, filed Aug. 14, 2020. The provisional application identified above is hereby incorporated by reference in its entirety herein to provide continuity of disclosure.

### TECHNICAL FIELD

The present disclosure is directed generally to liquid containers and, more specifically, to portable liquid dispensers for personal use.

### BACKGROUND

In light of the infectious diseases, such as coronaviruses, influenza viruses, and other viral and bacterial infections, that plague communities, there is a continuing need for simple and effective strategies for preventing or reducing the likelihood of such infections. One such strategy involves frequent cleansing and/or sanitizing of hands and other body parts, as well as environmental surfaces with which users come into frequent contact. The ability to carry a sanitizer on one's person increases the ability to sanitize one's hands frequently and as needed throughout the day. However, conventional containers for sanitizing fluids often are difficult to carry in clothing pockets and usually are intended to be disposed of when emptied. Such shortcomings often result in sanitizers not being carried and used by those who might otherwise do so.

Consequently, there is a need for a liquid dispenser that can address one or more of these and other shortcomings.

### SUMMARY

The present disclosure encompasses liquid dispensers that can be carried on a user's person, in a user's clothing or personal bag that can be configured to hold and dispense a sanitizing liquid, and that can be refillable and reusable through multiple fillings. The present disclosure encompasses a liquid dispenser comprising a container body defining a container chamber therein, wherein the container body comprises a dispenser port in fluid communication with the container chamber, wherein the container body comprises a fill port in fluid communication with the container chamber; an atomizer operably connected to the container body, wherein the atomizer is in fluid communication with the dispenser port and the container chamber; a fill port closure operably connectable to the container body, wherein the fill port closure is movable from a fill-port-closure-open position opening the fill port to a fill-port-closure-closed position closing the fill port; and, a cover movably connectable with the container body, wherein the cover is movable from a cover-closed position enclosing the fill port and a coyer-open position exposing the fill port, wherein the fill port closure is enclosed by the cover and the container body in the fill-port-closure-closed position and the cover-closed position.

In one aspect, the container body can comprise a first merlon and a second merlon separated from the first merlon by a crenel, wherein the first merlon comprises a first merlon chamber, wherein the second merlon comprises a second merlon chamber, and wherein the container chamber comprises the first merlon chamber and the second merlon chamber. In another aspect, the liquid dispenser further can comprise a hinge connecting the cover to the container body. In a further aspect, the cover can be pivotally movable on the hinge from the cover-closed-position to the cover-open. position. In yet another aspect, the liquid dispenser further can comprise a magnet connecting the cover to the container body in the cover-closed position. in still a further aspect, the magnet can be attached to the cover. In another aspect, the fill port closure can comprise a thread, and wherein the fill port closure is threadably engaged to the fill port in the fill-port-closure-closed position. In a further aspect, the fill port closure can comprise a resilient body, and wherein the resilient body is disposed in the fill port in the fill-port-closure-closed position. In still another aspect, the fill port can be aligned in the bottom wall of the container body. In yet another aspect, the cover can enclose the bottom wall of the container body in the cover-closed-position.

The present disclosure also encompasses a liquid dispenser comprising a container body defining a container chamber therein, wherein the container body comprises a dispenser port in fluid communication with the container chamber, wherein the container body comprises a fill port in fluid communication with the container chamber, wherein the dispenser port is formed within a first wall of the container body and the fill port is formed within a second wall of the dispenser body; an atomizer operably connected to the dispenser port, wherein the atomizer is in fluid communication with the container chamber; a fill port closure operably connectable to the container body, wherein the fill port closure is realignable between a fill-port-closure closed position wherein the fill port is closed and a fill-port-closure-open position wherein the fill port is open; and, a cover movably connectable with the container body, wherein the cover is realignable between a cover-closed-position wherein the fill port is enclosed and a cover-open position wherein the fill port is exposed.

In one aspect, the cover can be hingedly mounted to the container body. In another aspect, the container body further can comprise a first merlon and a second merlon separated from the first merlon by a crenel, wherein the first merlon comprises a first merlon top wall and the second merlon comprises a second merlon top wall, wherein the crenel comprises a crenel top wall, and wherein the dispenser port is aligned in the crenel top wall. In a further aspect, the cover can be detachable from the container body in the cover-open position. In yet another aspect, the cover can comprise a magnet, and wherein the magnet connects the cover to the container body in the cover-closed position. In still a further aspect, the fill port closure can comprise a thread formed thereon. and wherein the fill port closure is threadably connected to the fill port in the fill-port-closure-closed position. in another aspect, the fill port closure can comprise a resilient body, and wherein the resilient body frictionally engages the fill port in the fill-port-closure-closed position. In a further aspect, the second wall can be the bottom wall of the container body. In yet another aspect, the container body further can comprise a skirt depending from the bottom wall.

The present disclosure further encompasses a liquid dispenser comprising a container body defining a container chamber, wherein the container body comprises the top wall and a bottom wall opposing the top wall, wherein the container body comprises a dispenser port disposed in the top wall, wherein the dispenser port is in fluid communication with the container chamber, wherein the container body comprises a fill port disposed in the bottom wall, wherein the fill port is in fluid communication with the container chamber, and wherein the container body comprises a skirt depending from the bottom wall; a fill port closure operably connectable to the fill port, wherein the fill port closure is movable between. a fill-port-closure-open position opening the fill port and a fill-port-closure-closed position dosing the fill port; and, a cover magnetically connectable to the container body, wherein the cover comprises a magnet, wherein the cover is movable between a cover-open position exposing the fill port and a cover-closed position covering the fill port, and wherein the magnet magnetically engages the container body in the cover-closed position.

These and other aspects of the present disclosure are set forth in greater detail below and in the drawings for which a brief description is provided as follows.

### BRIEF DESCRIPTION OF THE DRAWINGS

FIG. 1 is a perspective view of a liquid dispenser encompassing aspects of the present disclosure, wherein the liquid dispenser is in a cover-closed position with the cover enclosing the fill port.

FIG. 2 is a front view of the liquid dispenser shown in FIG. 1 in a cover-closed position.

FIG. 3 is a front cross-sectional view liquid dispenser shown in FIG. 1 in a fill-port-closure-closed position, wherein the fill port is closed by the fill port closure, and a cover-closed position, wherein the cover encloses the fill port and the fill port closure.

FIG. 4 is a front view of the liquid dispenser of FIG. 2 with the port closure in a fill-port-closure-closed position and the cover in a cover-open position with the cover aligned exposing the fill port and the fill port closure.

FIG. 5 is a side view of the liquid dispenser shown in FIG. 4 with the fill port closure in the fill-port-closure-closed position and the cover in the cover-open position in a cover-open position with the cover pivoted on the hinge to a fully open alignment.

FIG. 6 is a perspective view of the top section of the container body of the liquid dispenser of FIG. 1 with the remainder of the liquid dispenser removed from the top section.

FIG. 7 is a bottom view of the top section of the container body shown in FIG. 6,

FIG. 8 is a cross-sectional view of the top section of the container body shown in FIG. 6.

FIG. 9 is a perspective view of an atomizer cap of the atomizer of the liquid dispenser shown in FIG. 1 with remainder of the atomizer and the liquid dispenser removed from the atomizer cap.

FIG. 10 is perspective view of a middle section the container body of the liquid dispenser shown in FIG. 1 with the remainder of the container body and the liquid dispenser removed from the middle section.

FIG. 11 is a perspective view of a bottom wall of the container body of the liquid dispenser of FIG. 1 with the remainder of the container body and the liquid dispenser removed from the bottom wall.

FIG. 12 is a side elevation view of the bottom wall shown in FIG. 11.

FIG. 13 is a perspective view of the fill port closure encompassing aspects of the present disclosure and shown in FIG. 3 in which the fill port closure is shown in a fill-port-closure-closed position closing the fill port.

FIG. 14 is a perspective view of a hinge piece and a hinge pin operably engaged with the hinge piece, wherein the hinge piece and hinge pin encompass aspects of the present disclosure and can be operably connected to the cover shown in FIG. 15 and the container body of the liquid dispenser shown in FIG. 1.

FIG. 15 is a perspective view of a cover encompassing aspects of the present disclosure and shown in FIG. 3 in which the cover is shown in a cover-closed position enclosing the fill port

FIG. 16 is an exploded view of an atomizer encompassing aspects of the present disclosure and shown in FIG. 3 operably connected to the container body of the liquid dispenser.

FIG. 17 is a perspective view of another liquid dispenser encompassing aspects of the present disclosure, wherein the liquid dispenser is aligned in a cover-closed position.

FIG. 18 is a cross-sectional view of the liquid dispenser shown in FIG. 18, wherein the fill port closure is aligned in a fill-port-closure-closed position, wherein the fill port is closed by the fill port closure, and the cover is aligned in a cover-closed position, wherein the fill port and the fill port closure are enclosed by the cover.

FIG. 19 is a front view of the liquid dispenser shown in FIG. 17 with the fill port closure removed from the fill port and in a fill-port-closure-open position, wherein the fill port is opened, and the cover removed from the container body and in a cover-open position, wherein the fill port is exposed.

FIG. 20 is a perspective view of the cover of the liquid dispenser shown in FIG. 17 with the cover removed from the remainder of the liquid dispenser.

FIG. 21 is a perspective view of the atomizer cap cover of the liquid dispenser shown in FIG. 17 with the atomizer cap cover removed from the remainder of the liquid dispenser.

FIG. 22 is a perspective view of the bottom wall of the container body of the liquid dispenser shown in FIG. 17 with the bottom wall removed from the remainder of the liquid dispenser.

FIG. 23 is a perspective view of the liquid dispenser shown in FIG. 17 with the fill port closure and the cover removed, thereby exposing the open fill port disposed in the bottom wall of the container body of the liquid dispenser.

### DETAILED DESCRIPTION

The present disclosure encompasses refillable and reusable compact liquid dispensers that can be used to carry, contain and dispense sanitizing liquids, as well as other liquids for personal use. The liquid dispensers encompassed by the present disclosure can comprise low profile configurations that allow for the liquid dispensers to be carried easily in and removed from clothing pockets, handbags, briefcases, backpacks and the like. The low profile configurations of the liquid dispensers encompassed by the present disclosure can comprise container bodies and covers with smooth outer surfaces, and rounded edges. The liquid dispensers can be free of components that protrude beyond the smooth outer surfaces, so as to reduce the likelihood of the liquid dispenser catching on clothing when being inserted into or removed from clothing pockets or other carrying containers. The present disclosure refers in detail below to various aspects of liquid dispensers, which are illustrated in the accompanying drawings. Wherever possible, the application uses the same reference numbers throughout the drawings to refer to the same or similar items.

As used herein, the singular forms of “a,” “an,” and “the” encompass the plural forms thereof unless otherwise indicated. As used herein, the phrase “at least one” includes all numbers of one and greater. As used herein, the term “and/or” refers to one or all of the listed elements or a combination of any two or more of the listed elements. As used herein, the phrase “integrally formed” means formed as a single, unitary body.

Some components of the liquid dispensers, including, but not limited to, the container bodies, the covers, the fill port closures, and the atomizer cap covers, encompassed by the present disclosure can be constructed from durable materials such as metals, composites, and polymers that can tend to resist chemical reaction with or degradation by sanitizing liquids or other liquids to be stored and dispensed from the liquid dispensers. The metals from which the liquid dispensers can be made include, but are not limited to, zinc, aluminum, steel, and various alloys thereof. The durable materials from which some liquid dispensers encompassed by the present disclosure can be made can allow for the extended use of the liquid dispensers through repeated filling and dispensing cycles.

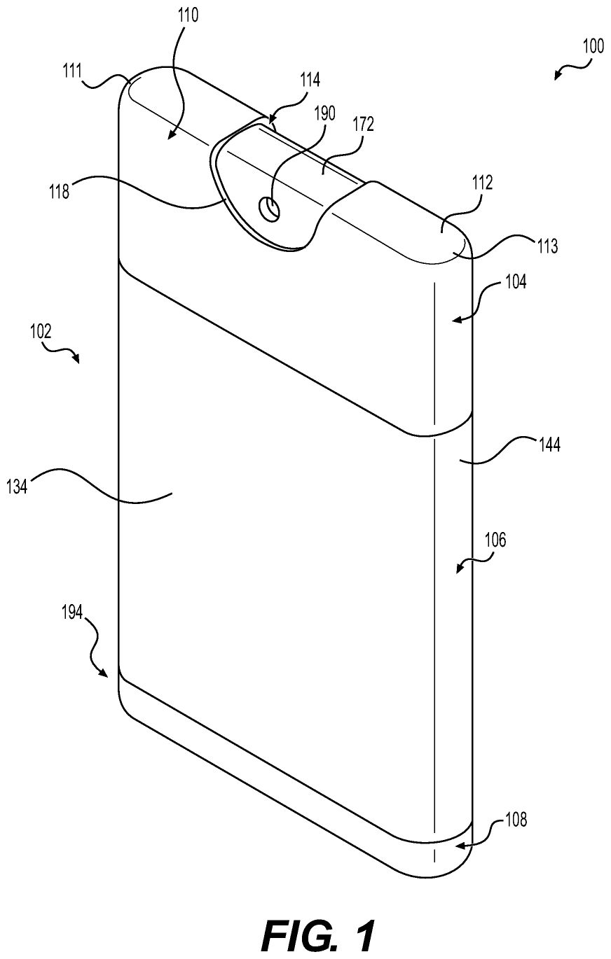

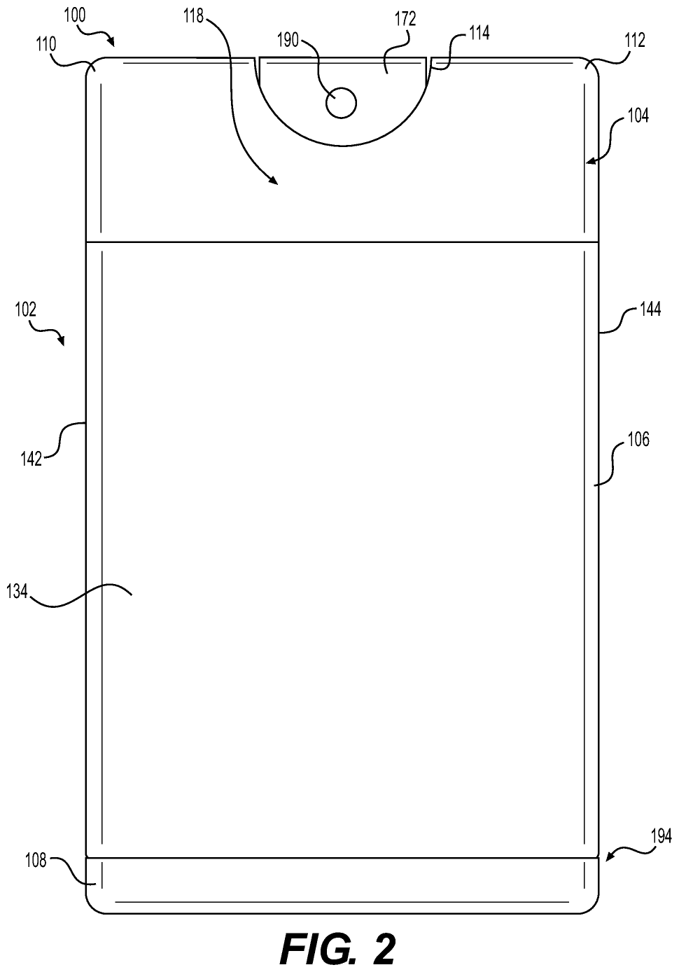

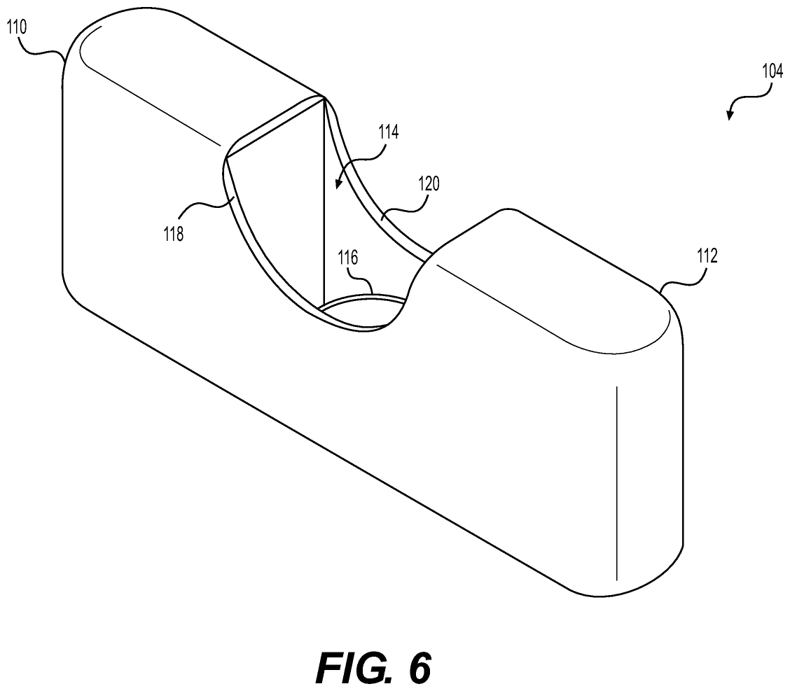

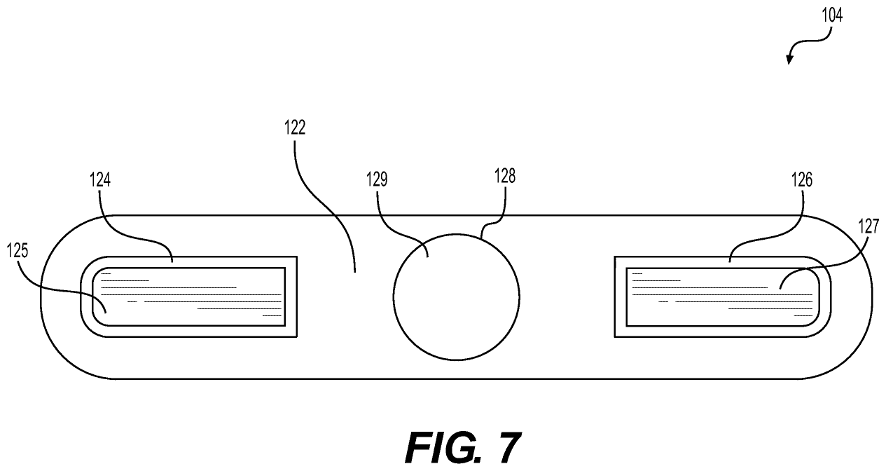

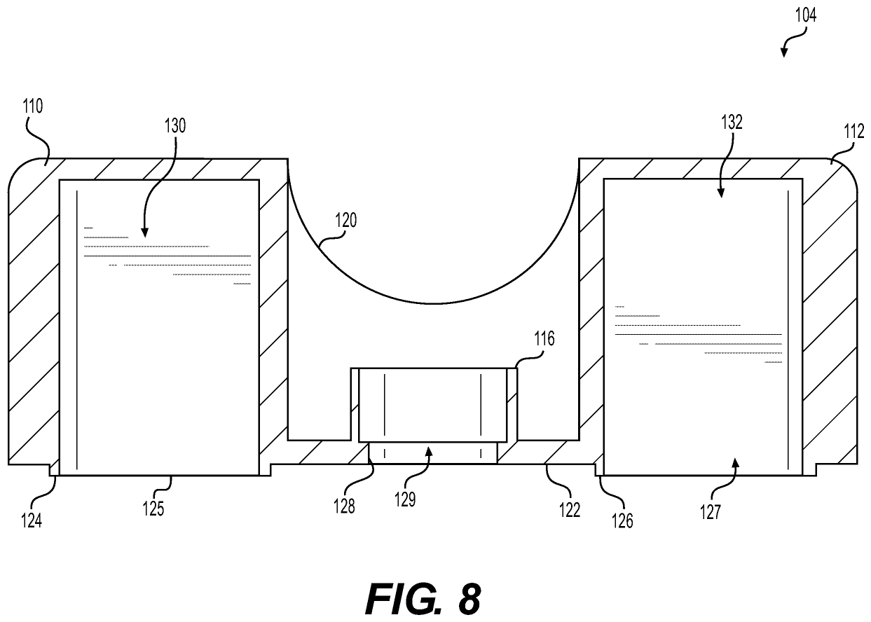

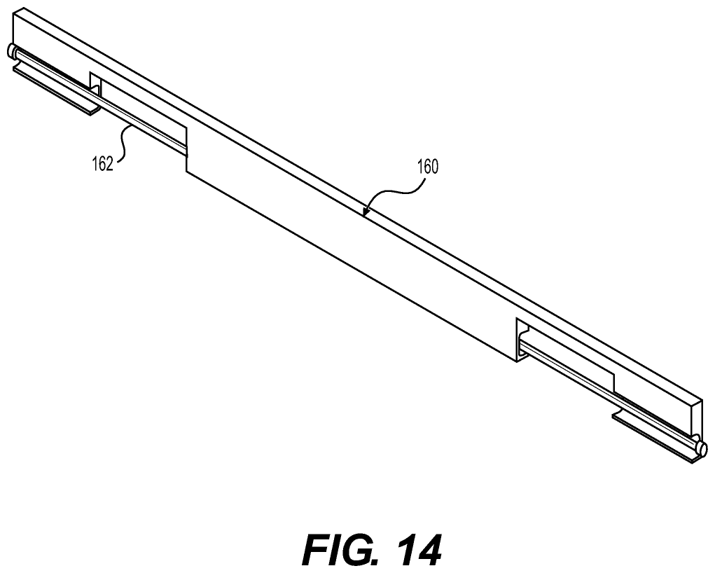

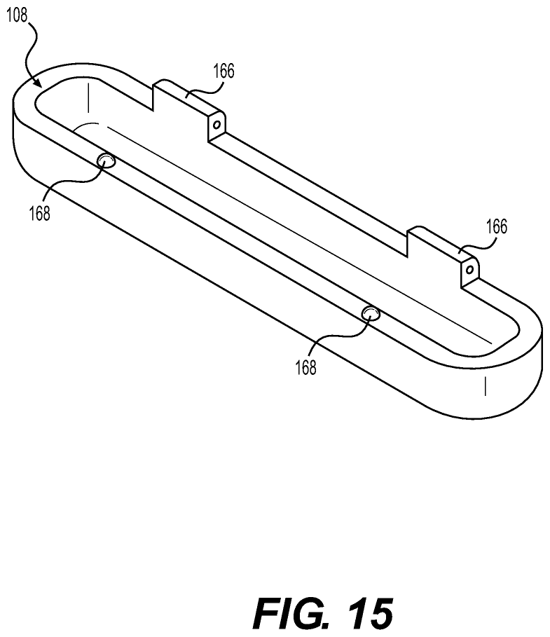

FIGS. 1-16 illustrate liquid dispenser 100 and parts thereof, all encompassing aspects of the present disclosure. As shown in FIGS. 1-3, the liquid dispenser 100 comprises a container body 102 that defines a container chamber 146 for receiving, storing, and dispensing liquid, such as a sanitizing liquid or other suitable fluid. An atomizer 170 is operably connected to and extends through a dispenser port 129 that is both defined in the container body 102 and in fluid communication with the container chamber 146, thereby allowing the atomizer 170 to be in in fluid communication with both the container chamber 146 and the outside environment of the liquid dispenser 100 to allow for the liquid contained in the container chamber 146 to be sprayed out of the liquid dispenser 100 in a fine mist through the atomizer cap opening 190 of the atomizer 170.

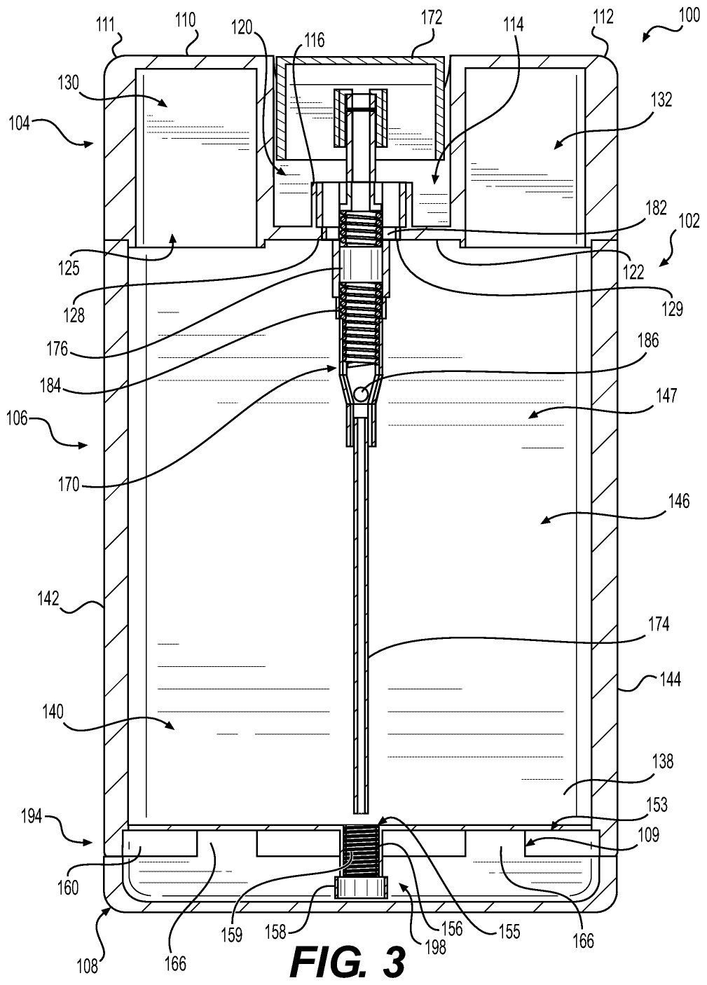

The liquid dispenser 100 can be filled and refilled with liquid through the fill port 155 disposed in a wall of the container body 102. As shown in FIG. 3, the fill port 155 can be disposed in the bottom wall 153 of the container body 102 The present disclosure also encompasses liquid dispensers having fill ports aligned in alternative positions on the container body.

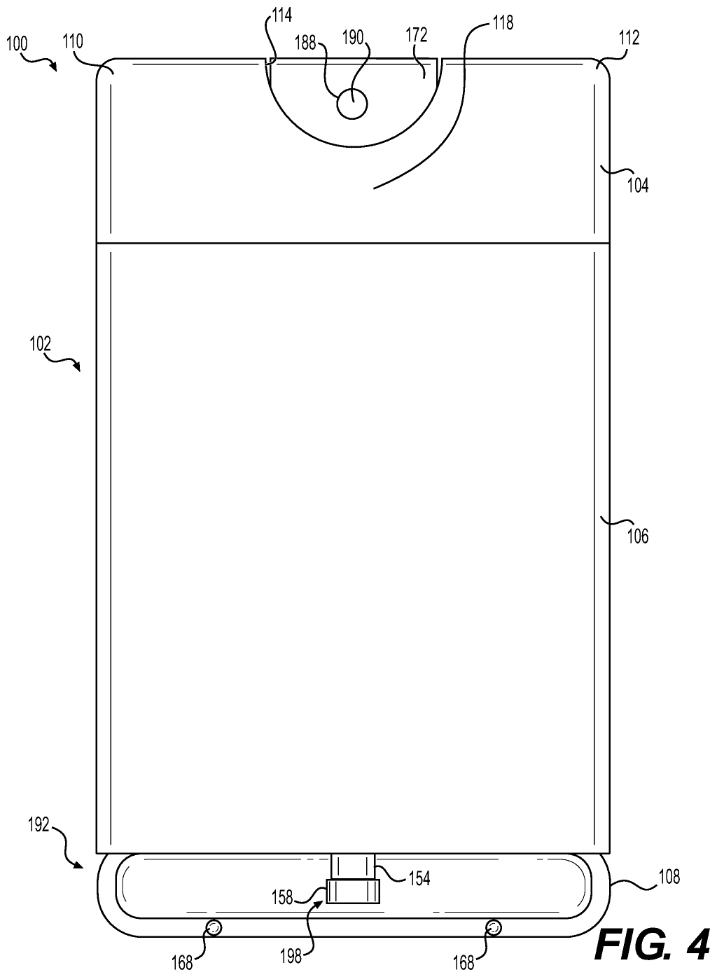

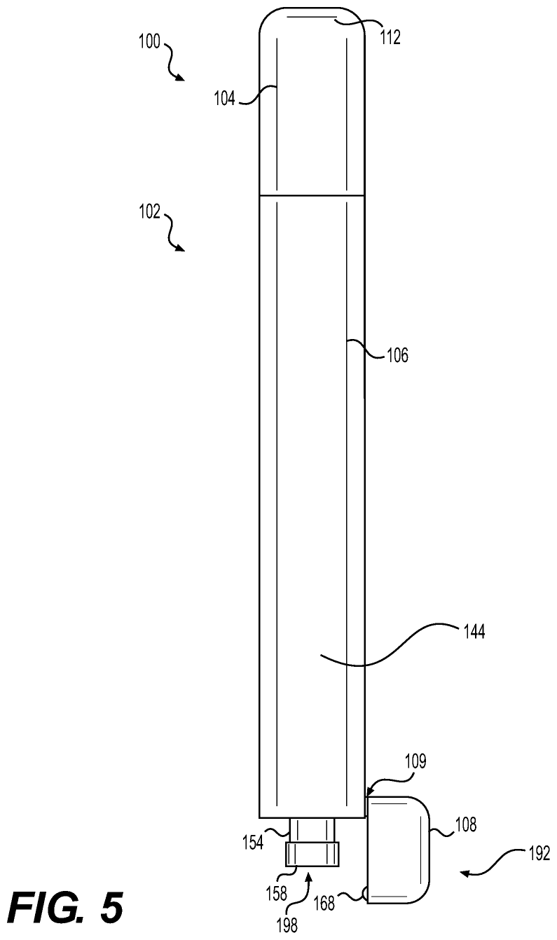

A cover 108 is pivotally connected by a hinge assembly to the container body 102 and is configured to pivot alternatively between a cover-open position 192, in which the fill port 155 and/or the fill port closure 158 are exposed to the outside environment of the liquid dispenser 100, as shown in FIGS. 4 and 5, and a cover-closed position 194, in which the fill port 155 and/or the fill port closure 158 are enclosed by the cover 108 and the remainder of the container body 102, as shown in FIGS. 1, 2 and 3.

As shown in the FIGS. 3 and 5, the cover 108 is hingedly connected to the container body 102 by a hinge 109. The hinge 109 comprises a hinge piece 160 that includes hinge piece knuckles. The hinge 109 is mounted to the container body 102. A plurality of cover hinge knuckles 166 are formed on the cover 108 and are aligned with the hinge piece 160. A hinge pin 162 extends through the hinge piece knuckles and the cover hinge knuckles 166, thereby acting as a pivot point for the cover 108 to pivotally move back and forth between the cover-closed position 194 and the cover-open position 192. The hinge 109 maintains the cover 108 in a proper alignment such that, when the cover 108 is pivoted on the hinge 109, the cover 108 is aligned to cover the bottom end of the container body 102 in the cover-dosed position 194.

The container body 102 comprises a main section 106, a top section 104 attached to the top end of the main section 106, and a bottom wall 153 attached to the bottom end of the main section 106. The container body 102 comprises a front wall 134, a rear wall 140 opposing the front wall 134, a first side wall 142 extending between and connected to the front wall 134 and the rear wall 140, and a second side wall 144 opposing the first side wall 142 and extending between and connected to the front wall 134 and the rear wall 140. The front wall 134, the rear wall 140, the first side wall 142, and the second side wall 144 are formed by portions of both the top section 104 and the main section 106. The front wall 134 and the rear wall 140 are shown as being aligned substantially parallel to each other to facilitate the stowing of the liquid dispenser 100 in a pocket of a user's clothing or bag. In one aspect, both the front wall 134 and the rear wall 140 can be substantially flat to provide a reduced profile for the liquid dispenser 100. Both the first side wall 142 and the second side wall 144 are illustrated as being curved and convex, thereby eliminating sharp edges along the lengths of the front wall 134 and the rear wall 140.

Both the top section 104 and the bottom wall 153 can be attached to the main section 106 by welding, soldering, adhesive or other known attachment means, and, alternatively, can be integrally formed with the main section 106, thereby making the container body 102 a unitary body in either case.

The container body 102 comprises a first shoulder 111 formed on a first merlon 110 and a second shoulder 113 formed on a second merlon 112 and opposing the first shoulder 111. The first merlon 110 is separated from the second merlon 112 by a crenel 114 disposed therebetween. The crenel 114 comprises a crenel top wall 122 that is spaced apart from, and can be parallel to, the top walls of the first merlon 110 and the second merlon 112 and is aligned proximal to the bottom wall 153, whereas the top walls of the first merlon 110 and the second merlon 112 are aligned distal to the bottom wall 153. The first merlon 110 defines a first merlon chamber 130 therein, and the second merlon 112 defines a second merlon chamber 132 therein. Each of the first merlon chamber 130 and the second merlon chamber 132 are in fluid communication with the main section chamber 147 defined by the main section 106. The container chamber 146 can comprise any one or more of the first merlon chamber 130, the second merlon chamber 132 and the main section chamber 147. The first merlon chamber 130 and the second merlon chamber 132 are aligned distal to the bottom wall 153, whereas the main section chamber 147 is aligned proximal to the bottom wall 153.

The dispenser port 129 is aligned at the crenel 114 and extends through the crenel top wall 122. The dispenser port 129 can comprise a circular sidewall 116 extending outward from the crenel top wall 122. The dispenser port 129 also comprises a dispenser port lip 128 formed in the crenel top wall 122 and which defines a dispenser port opening 129 that is in fluid communication with the container chamber 146, and, as shown in FIG. 3, can be in fluid communication with the main section chamber 147.

As shown in FIGS. 1, 3, 6, and 8, the top section 104 comprises a front collar 118 and a rear collar opposing the front collar 118. Each of the front collar 118 and the rear collar 120 extends between the first merlon 110 and the second merlon 112 and comprises an arcuate top wall that is aligned lower than the first top wall 111 and the second top wall 113 of each of the first merlon 110 and the second merlon 112 and higher than the crenel top wall 122. The front collar 118 and the rear collar 120, along with the opposing faces of the first merlon 110 and the second merlon 112, form parts of the respective front all 134 and the rear wall 140.

As shown in FIGS. 3, 8, and 9, the top section 104 comprises the crenel top wall 122, which extends between a portion of the front wall 134, a portion of the rear wall 140, a portion of the first side wall 142 and a portion of the second side wall 144. The top section 104 comprises a first merlon lip 124 that defines a first merlon opening 125 and a second merlon lip 126 that defines a second merlon opening 127. The first merlon chamber 130 is in fluid communication with the main section chamber 147 through the first merlon opening 125, and the second merlon chamber 132 is in fluid communication with the main section chamber 147 through the second merlon opening 127. The dispenser port 129 is disposed between the first merlon opening 125 and the second merlon opening 127.

The cover 108 is aligned adjacent the bottom end of the container body 102 and covers the bottom wall 153 of the container body 102 in the cover-closed position 194. The cover 108 is movable between the cover-closed position 194, shown in FIGS. 1-3, and the cover-open position 192, shown in FIGS. 4 and 5. In the cover-closed position 194, the fill port 155 is enclosed between the cover 108 and the container body 102; and, when the fill port closure 158 is in the fill-port-closure-closed position 198, as shown in FIG. 3, the fill port closure 158 is also enclosed by the cover 108 and the container body 102 in the cover-closed position 194. In the cover-open position 192, the bottom wall 153 and the fill port 155 are exposed, with the fill port 155 accessible for refilling the container chamber 146.

The cover 108 is elongated and configured to have similar dimensions to the container body 102. The width of the cover 108 is approximately equal to the width of the container body 102, thereby allowing the cover 108 to cover the entire bottom wall 153 of the container body 102 when the cover 108 is in the cover-closed position 194. The height of the cover 108 is greater than the combined height of the fill port 155 and the fill port closure 158, when the fill port closure 158 is in the fill-port-closure-closed position 198. The cover 108 has rounded lower corners for minimizing the catching of the cover 108 on clothing when placed into a clothing pocket.

As shown in FIGS. 4, 5 and 15, the cover 108 comprises a plurality of magnets that magnetically connect the cover 108 to the container body 102. The cover 108 comprises a pair of magnets 168 disposed on the top surface thereon. Each magnet 168 attached to the cover 108 can be either flush with the top wall of the cover 108 or project upward therefrom. Each magnet 168 is aligned to magnetically engage the container body 102 directly, if the container body 102 is formed of a ferromagnetic material, or a metal component insert aligned on or in the container body 102, at or proximal to the container bottom wall 153 so as to retain the cover 108 in the cover-closed position 194. A user can apply sufficient amount of force to overcome the magnetic attraction of the magnets 168 connecting the cover 108 to the container body 102 open the cover 108.

As shown in FIGS. 3 and 13, the fill port closure 158 comprises a threaded shaft 159 and a cap extending from the shaft 159. A seal ring 161 is aligned on the threaded shaft 159 to provide a liquid-tight seal between the fill port closure 158 and the fill port 155 when the fill port closure 158 is in the fill-port-closure-closed position 198.

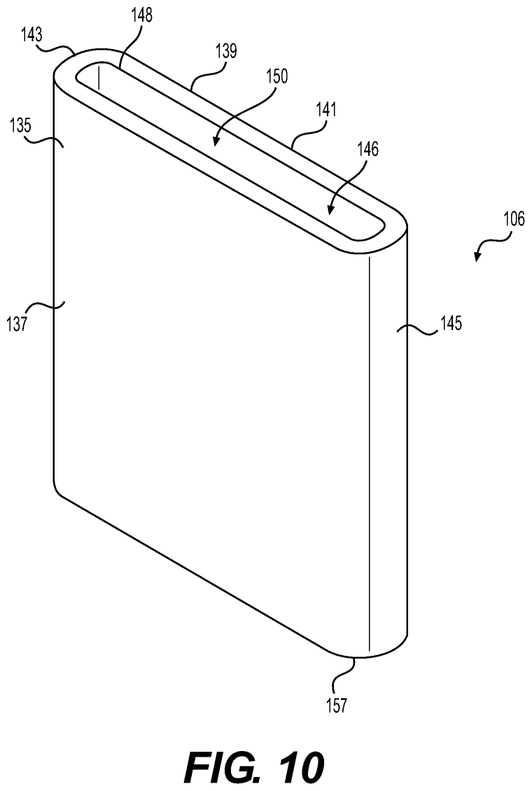

As shown in FIG. 10, the main section 106 of the container body 102 can comprise a main section front wall 135, a main section rear wall 141 opposing the main section front wall 135, a main section front face 137 defined by the main section front wall 135, a main section rear face 139 defined by the main section rear wall 141, a main section first side wall 143 extending between the main section front wall 135 and the main section rear wall 141, and a main section second side wall 145 opposing the main section first side wall 143 and also extending between the main section front wall 135 and the main section rear wall 141. The main section 106 defines a portion of the container chamber 146 therein. A main section top lip 148 defines a main section top opening 150 in the top of the main section 106. A main section bottom opening 157 defined in the bottom of the main section 106 can be substantially enclosed b the bottom wall 153, which can be welded, adhered, otherwise connected or integrally formed with the main section 106. The widths of the main section front wall. 135 and the main section real wall 141 are wider than the widths of the main section first side wall 143 and the main section second side wall 145. The transition area between the main section front and rear walls 135 and 141 and the main section first and second side walls 143 and 145 can be rounded, as shown in FIG. 10 so as to facilitate the insertion and removal of the liquid dispenser 100 from a clothing pocket of the user. The main section 106 can be constructed as a unitary piece, as shown in FIG. 10.

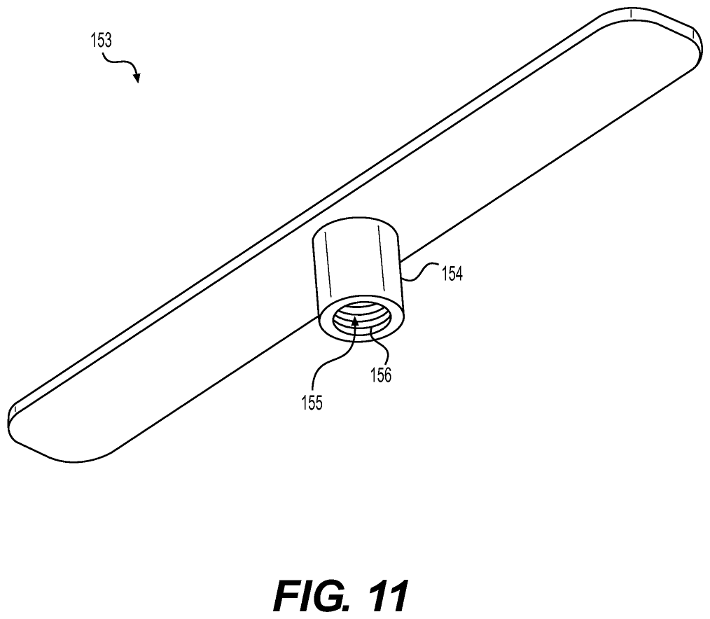

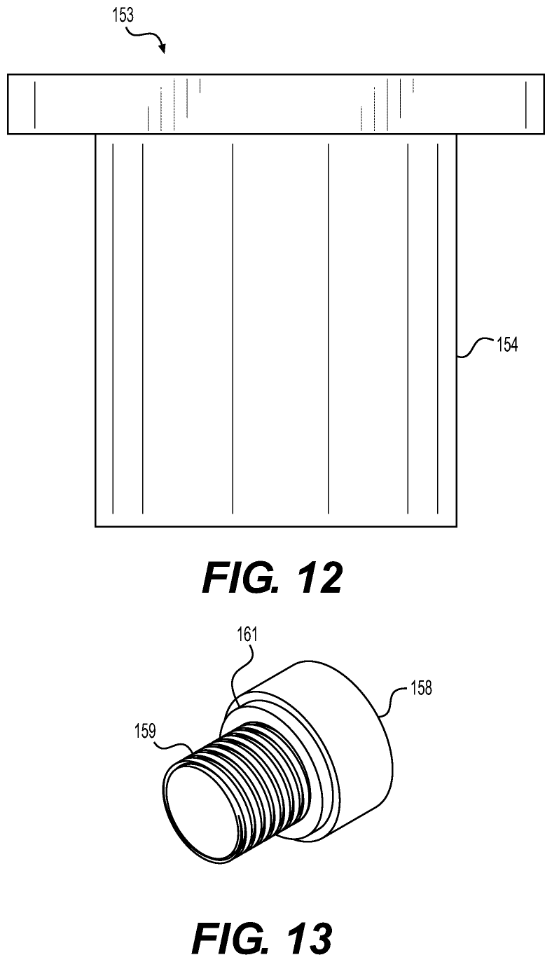

As shown in FIGS. 11 and 12, the bottom wall 153 is elongated and configured to fit into and be secured in the main section bottom opening 157 of the main section 106. The bottom wall 153 also comprises the fill port 155, which is in fluid communication with the container chamber 146. The fill port 155 comprises a fill port neck 154 that extends from the bottom wall 153. The fill port neck 154 is cylindrical and comprises a neck thread 156.

The fill port 155 is configured to receive the fill port closure 158 shown in FIG. 13. The fill port closure 158 comprises a closure thread 159 that can threadably engage the neck thread 156 of the fill port 155. A sealing ring 161 can be operably connected to the fill port closure 158 to facilitate the liquid-tight seal between the fill port closure 158 and the fill port 155 when the fill port closure 158 is in the fill-port-closure-closed position 198.

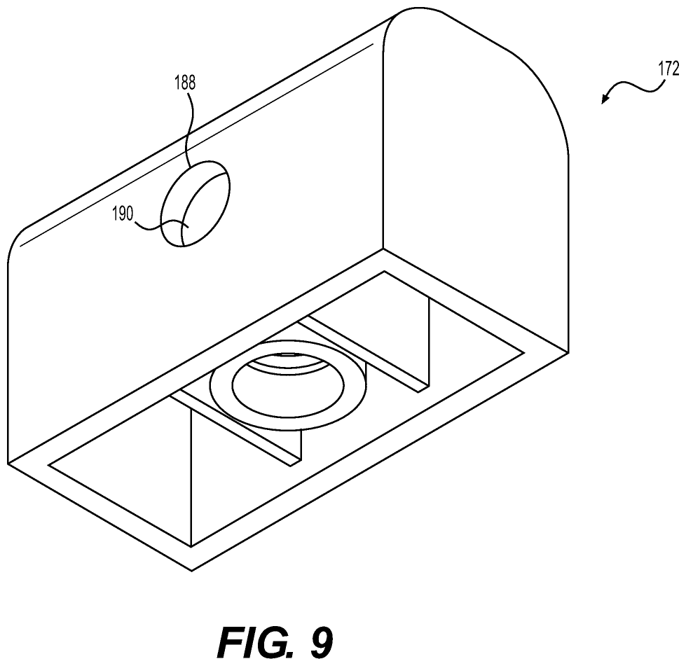

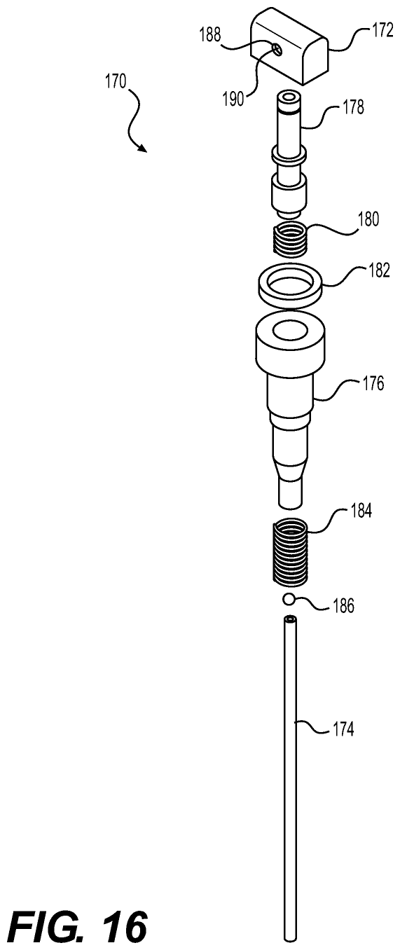

As shown in FIGS. 3 and 16, the liquid dispenser 100 comprises an atomizer 170 operably connected to the container body 102 and in fluid communication with the container chamber 146. The atomizer 170 comprises a dipper 174, at least a portion of which is disposed inside of the container chamber 146 and which can extend into the main section chamber 147. The dipper 174 is illustrated as an elongated tube that has a top opening and a bottom opening defined by the tube body. The dipper 174 is in fluid communication with the container chamber 146 through the bottom opening thereof. The dipper 174 is operably connected to an atomizer body 176, which defines a conduit therethrough and defines a body top opening and a body bottom opening. The dipper 174 can be at least partially aligned within the conduit of the atomizer body 176. An atomizer ball 186 is operationally aligned within the atomizer body 176 and is aligned upstream of the dipper 174. The atomizer ball 186 is disposed within a frustoconical section of the conduit of the atomizer body 176 upstream of a feed inlet of the atomizer body 176, thereby allowing the atomizer ball to at least partially block the flow of liquid up through the atomizer body 176. A first atomizer spring 184 is operably aligned within the atomizer body 176 upstream of the atomizer ball 186. A depressor body 178 is operably connected to the atomizer body 176, and a portion of the depressor body 178 can be aligned within the conduit of the atomizer body 176. The depressor body 178 comprises a conduit extending therethrough and which is in fluid communication with both the atomizer body 176 and the dipper 174 during at least a portion of the period in which the atomizer 170 is operational. A portion of the depressor body 178 is aligned within the conduit of the atomizer body 176 between the first atomizer spring 184 and a second atomizer spring 180 and can engage each of the first and second atomizers springs 184 and 180. The depressor body 178 is operably connected to an atomizer cap 172. The atomizer cap 172, as shown in FIG. 9, comprises an atomizer cap lip 188 formed in an outer wall thereof and which defines an atomizer cap opening 190. The atomizer cap opening 190 is in fluid communication with the conduit of the depressor body 178.

The atomizer 170 is connected to and at least partially disposed in the dispenser port 129, with a portion of the atomizer 170 aligned outside of the container body 102 and at least another portion of the atomizer disposed within the container body 102. A ring 182 is operably connected to the atomizer body 176 to secure the atomizer 170 in the dispenser port 129. The atomizer cap 172 is disposed in or slightly above the crenel 114 between the first merlon 110 and the second merlon 112. A portion of the atomizer cap 172 can be disposed between the front collar 118 and the rear collar 120 so as to tend to minimize the profile of the atomizer 170. In one aspect, the atomizer cap 172 can be aligned flush with or below the top walls of the first merlon 110 and the second merlon 112 such that those top walls define the most distal edges of the container body relative to the bottom surface of the cover 108.

Liquid stored in the container chamber 146 can be dispersed through the atomizer 170 by depressing the atomizer top cap 172. When the atomizer top cap 172 is depressed at least one of the first atomizer spring 184 and the second atomizer spring are depressed and liquid is thereby drawn from the container chamber 146 ups through, in turn, the conduits in each of the dipper 174, the atomizer body 176, and the depressor body 178, and then out through the atomizer cap opening 190. The liquid is thereby sprayed in a fine mist outward from the liquid dispenser 100, and, where the liquid is a sanitizing liquid, can be sprayed on hands or other parts of a user's body or other surfaces to sanitize those areas.

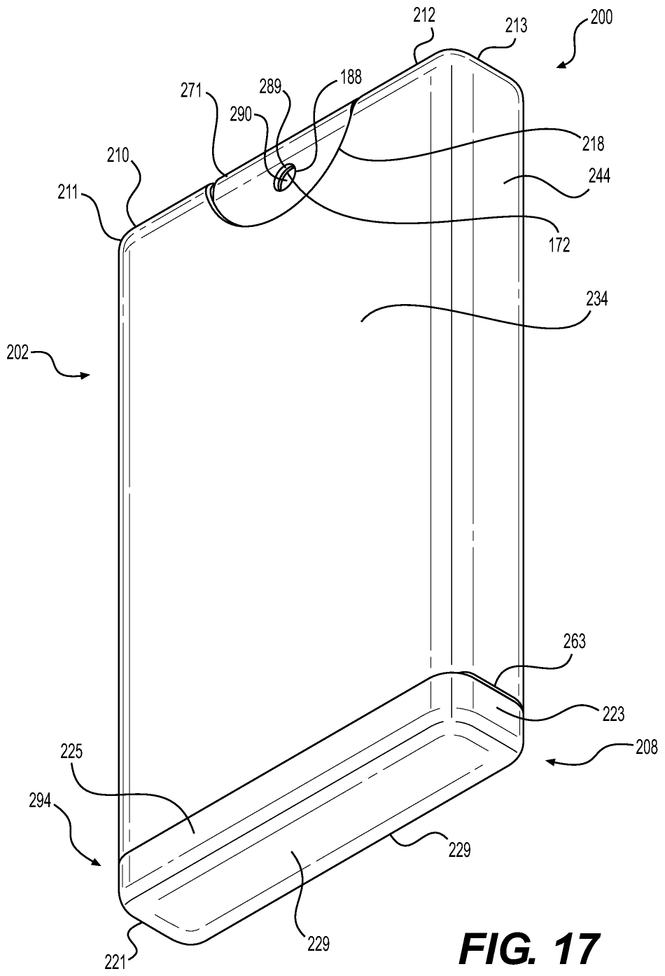

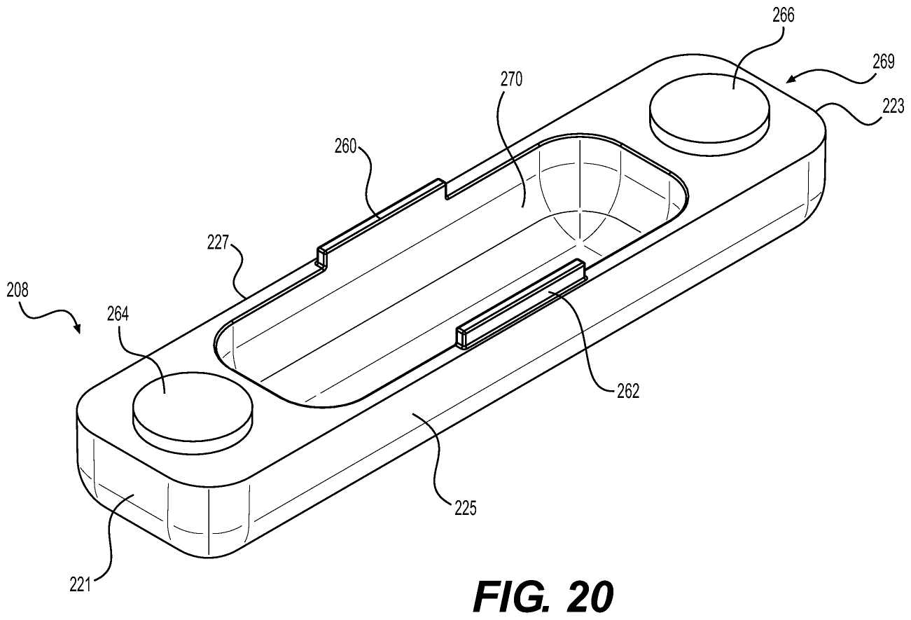

FIGS. 17-23 illustrate another liquid dispenser 200 encompassing aspects of the present disclosure. Unlike the liquid dispenser 100, the liquid dispenser 200 comprises a cover 208 that can be completely detached from the container body 202. Furthermore, the container body 202 is unitary in design with the upper portion and middle portions thereof being integrally formed together. Also, unlike the liquid dispenser 100, the liquid dispenser 200 does not comprise a hinge connecting the cover 208 to the container body 202. Instead, the cover 208 is completely detachable from the container body 202 the liquid dispenser 200. The cover 208 comprises a plurality of magnets that magnetically connect the cover 208 to the container body 202 when the cover 202 is in a cover-closed position 294. The cover 208 is completely detached from the container body 202 in the cover-open position 292. The container body 202 defines a container chamber 246 therein in which a liquid, such as a sanitizing liquid, can be contained and from which the liquid can be dispensed.

As shown in FIGS. 17, 18 and 19, the container body 202 comprises a front wall 234, a rear wall 240 opposing the front wall 234, a first side wall 242 extending between the front wall 234 and the rear wall 240, and a second side wall 244 opposing the first side wall 242 and extending between the front wall 234 and the rear wall 240. The container body 202 comprises a first merlon 210, a second merlon 212, and a crenel 214 formed between the first merlon 210 and the second merlon 212. The first merlon 210 comprises a first merlon top wall 211, and the second merlon 212 comprises a second merlon top wall 213. The first merlon 210 and the second merlon 212 define a pair of shoulders for the container body 202 with the first merlon top wall 211 and the second merlon top wall 213 offset from the crenel top wall 215. Adjacent the crenel 214 is a front collar 218 and a rear collar 220 opposing the front collar 218. The front collar 218 and the rear collar 220 extend between the first merlon 210 and the second merlon 212, and above the crenel top wall 215. Each of the front collar 218 and the rear collar 220 comprise a semicircular top edge.

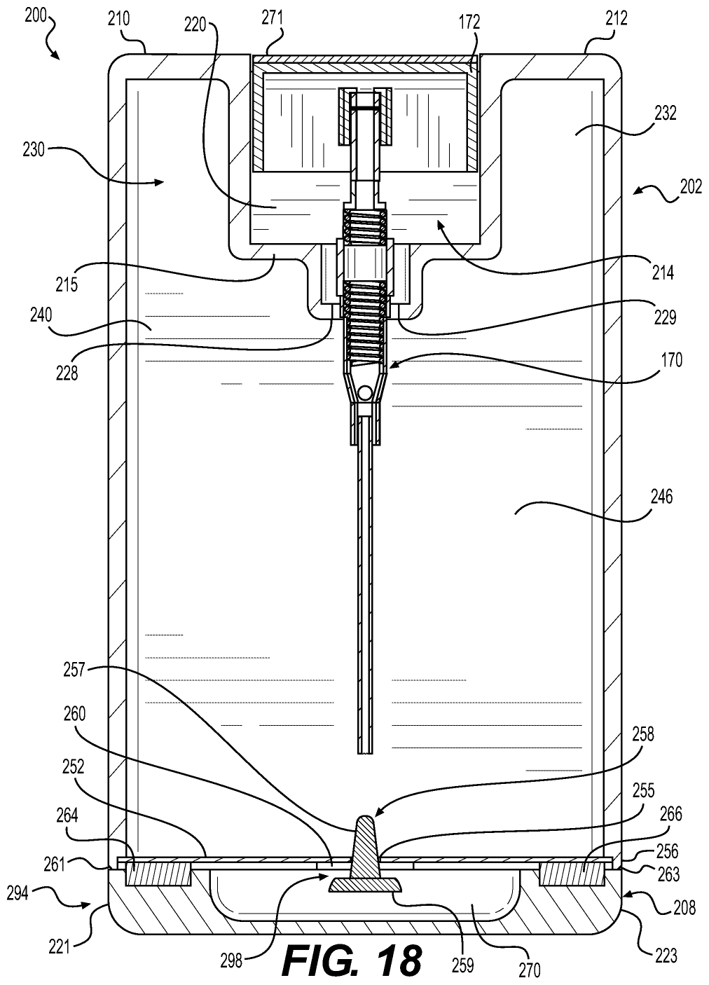

A dispenser port 229 is disposed in the crenel top wall 215. As shown in FIG. 18, the dispenser port 229 is aligned in the mid section of the crenel top wall 215 and is in fluid communication with the container chamber 246. The atomizer 170 is operably connected to the container body 202 and disposed in the dispenser port 229, thereby allowing liquid contained within the container chamber 246 to be dispensed through the dispenser port 229 through the atomizer 170. As shown in FIG. 18, the first merlon 210 comprises a first merlon chamber 230 and the second merlon 212 comprises a second merlon chamber 232. The container chamber 246 comprises the first merlon chamber 230 and the second merlon chamber 232, thereby allow the entirety of the interior of the container body 202 to serve as a reservoir for liquid.

The container body 202 also comprises a bottom wall 252, as shown in FIG. 18. The bottom wall 252 is aligned opposing the first merlon top wall 211, the second merlon top wall 213 and the crenel top wall 215. The bottom wall 252. extends between the first side wall 242, the second side wall 244, the front wall 234 and the rear wall 240. The bottom wall 252 comprises a fill port 255, which defines a fill port opening 253 that is in fluid communication with the container chamber 246. The fill port 255 is in fluid communication with the container chamber 246. The fill port 255 is open in the fill-port-closure-open position 296 and closed in the fill-port-closure-closed position 298. In the fill-port-closure-open position 296, the container chamber 246 can be filled and/or refilled with liquid through the fill port 255. In the fill-port-closure-closed position 298, the fill port 255 is closed, thereby allowing liquid to be contained in the container chamber 246.

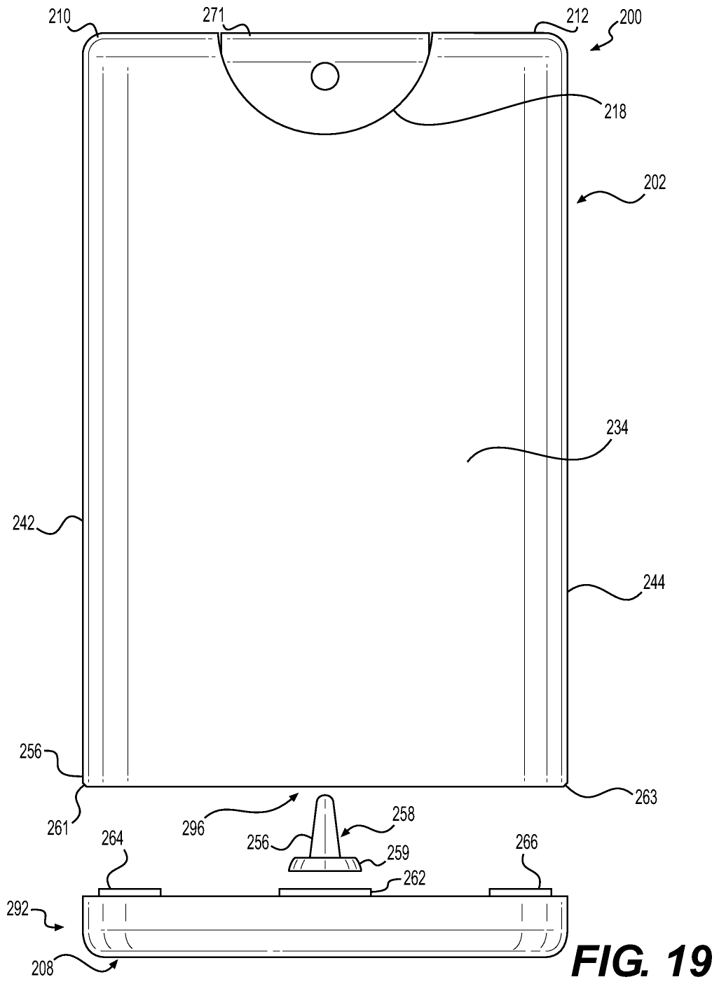

As shown in FIGS. 18 and 19, the liquid dispenser 200 comprises a fill port closure 258 that is operably connected to the fill port 255 in the fill-port-closure-closed position 298 and detached from the fill port 255 in the fill-port-open position 296, thereby exposing the fill port 255. The fill port closure 258 can be provided in the form of a plug comprising a fill port closure body 257 projecting from a fill port closure cap 259. The fill port closure body 257 can be provided in a frustoconical configuration, as shown in FIGS. 18 and 19. The fill port closure cap 259 can be wider than the base of the fill port closure body 257. In the fill-port-closure-closed position 298, the fill port closure 258 is frictionally engaged with the fill port 255 with the fill port closure body 257 extending through and sealing the fill port opening 253 and the fill port closure cap 259 positioned outside of and adjacent to the bottom wall 252 of the container body 202. The fill port closure 258 can comprise a resilient material, such as cork, rubber or another elastomeric polymer able to withstand extended contact with the liquid contained in the liquid dispenser 200. The fill port closure cap 259 and the rest of the fill port closure 258 is configured to be enclosed by the container body 202 and the cover 208 in the fill-port-closure-closed position 298 and the cover closed position 294.

As shown in FIGS. 17, 18 and 20, the cover 208 comprises a first cover side wall 221, a second cover side wall 223 opposing the first cover side wall 221, a cover front wall 225, a cover rear wall 227 opposing the cover front wall 225, and a cover bottom wall 229. The cover 208 comprises four rounded corners with one of the four rounded corners disposed between the adjacent walls. The cover 208 also comprises a cover well 270 defined therein and open to the top wall thereof. The fill port closure cap 259 can be aligned within the cover well 270 when the liquid dispenser)0 is in the fill-port-closure-closed position 298 and the cover closed position 294.

A plurality of guides projects upward from the top wall of the cover 208. The plurality of guides comprises a front guide 262 aligned adjacent the cover well 270 and the cover front wall 22, and a rear guide 260 aligned adjacent the cover well 270 and the cover rear wall 227. The front guide 262 and the rear guide 260 are aligned parallel to each other and project upward from the top wall of the cover 208. The front guide 262 and the rear guide 260 are configured as rectangular tabs, each of which has a width greater than its height.

The cover 208 also comprises a plurality of magnets 269 disposed thereon. The plurality of magnets 269 comprises a first magnet 264 and a second magnet 266, each of which face upward from the top wall of the cover 208. The first magnet 264 is aligned proximal to the first cover side wall 221 and distal to the second cover side wall 223. The second magnet 266 is aligned proximal to the second cover side wall 223 and distal to the first cover side wall 221. The first and second magnets 264 and 266 are magnetically attracted to the bottom wall 252 of the container body 202. The first and second magnets 264 and 266 magnetically connect the cover 208 to the container body 202 in the cover-closed position 294.

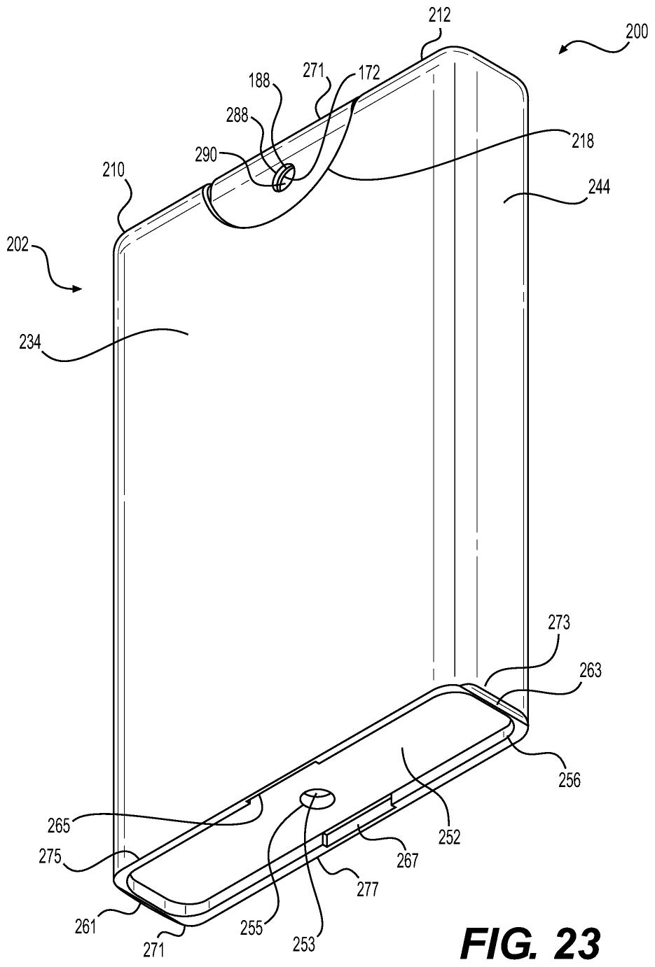

The container body 202, as shown in FIGS. 18 and 23, comprises a plurality of guide receivers formed thereon. The container body 202 comprises a front guide receiver 265 and a rear guide receiver 267 opposing the front guide receiver 265. The front guide receiver 265 and the rear guide receiver 267 are disposed in the skirt 256 and are aligned along medial lines of the front wall 234 and the rear wall 240, respectively. The front guide receiver 265 and the rear guide receiver 267 are arranged in the form of rectangular insets, channels or grooves sized and configured to receive the front guide 262 and the rear guide 260, when the cover 208 is brought into alignment with the container body 202 in the cover-closed position 294. The front and rear guides 262 and 260 and the front and rear guide receivers 265 and 267 are configured and aligned to engage each other when the cover 208 is being placed into the cover-closed position 294, thereby aligning the cover 208 in the proper orientation relative to the container body 202 so as to cover the bottom end of the container body 202.

The skirt 256 depends from the bottom of the container body 202 and projects beyond the bottom wall 252. The skirt 256 extends around the periphery of the walls of the container body 202. The skirt comprises a front skirt wall 275, a rear skirt wall 277 opposing the front skirt wall 275, a first skirt side wall 271 and a second skirt side wall 273 opposing the first skirt side wall 271. As shown in FIG. 23, a first notch 261 is formed on the first skirt side wall 271, and a second 263 is formed on the second skirt side wall 273. The first and second notches 261 and 263 extend to the lower edge of the skirt 256 and create points of access to the top surfaces of the cover 208 in the cover-closed position 294. A user of the liquid dispenser 200 can insert a fingernail or tool into each of the first and second notches 261 and 263 to contact the cover 208 and apply pressure thereto to remove the cover 208 from the container body 202.

When the cover 208 is in the cover-open position 292, the cover 208 is completely detached from the container body 202, thereby exposing the bottom wall 252, the fill port 155, and the fill port closure 258, if the fill port closure 258 is in the fill-port-closure-closed position 298. When the cover 208 is in the cover-closed position 294, the front guide 262 operably engages the front guide receivers 265, and the rear guide 260 operably engages the rear guide receiver 267, thereby arranging the cover 208 in the proper alignment relative to the container body 202. The first and second magnets 264 and 266 disposed on the cover 208 magnetically engage the bottom wall 252 of the container body 202, thereby connecting the cover 208 to the container body 202 and securing the cover 202 in that alignment until the user is ready to open the cover 208.

As shown in FIGS. 17, 18, 19, 21 and 23, the liquid dispenser 200 comprises an atomizer cap cover 271 that is connected to the atomizer cap 172. The atomizer cap cover 271 can be formed of the same material as the material from which the container body 202 is formed. For example, if the container body 202 is formed of aluminum, the atomizer cap cover 271 also can be formed of aluminum, thereby providing the liquid dispenser 200 with a consistent outer finish on all external parts thereof. Even when several of the parts of the atomizer 170, including the atomizer cap 172 are formed of a polymer, the liquid dispenser 200 can be given complete metallic finish by the incorporation of an atomizer cap cover 271 therein.

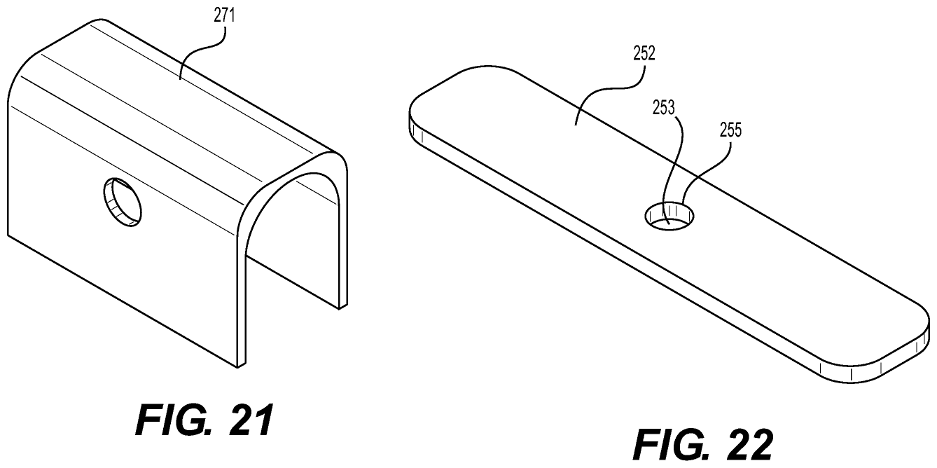

The atomizer cap cover 271 can comprise a continuous piece that is molded to fit over the top and front and back surfaces of the atomizer cap 172. The atomizer cap cover 271 comprises an atomizer cap cover front face, an atomizer cap cover rear face, and an atomizer cap cover top wall, each of which can be aligned adjacent to the respective cap front face, cap rear face and cap top wall. The atomizer cap cover 271 comprises an atomizer cap cover lip that defines an atomizer cap cover opening 290 that can be aligned with the atomizer cap opening 190 to allow liquid to be sprayed therethrough from the liquid dispenser 200. The atomizer cap cover 271 can be press fit onto the atomizer cap 172 to secure the atomizer cap cover 271 in place on the atomizer cap 172. As shown in FIG. 21, the atomizer cap cover 271 can have an arcuate type cross-section arising from the alignment of the atomizer cap cover front face, the atomizer cap cover rear face and the atomizer cap cover top wall.

The liquid dispensers 100 and 200 can be constructed in different versions that exhibit differing heights, widths and depths. In one aspect, the liquid dispenser 100 can exhibit, in the cover-closed position, an overall height of about 9.3 cm and an overall width of about 1.1 cm in which the main section main section 106 exhibits a height of about 6.7 cm, the top section 104 and exhibits a height of about 2 cm and the cover 108 exhibits a height of about 0.64 cm and with each of the top section 104, the main section 106 and the cover 108 exhibiting a width of about 1.1 cm. In another aspect, the liquid dispensers 100 and 200 can exhibit a width of about 5 cm, a depth of about 1.2 cm, a container body height of about 8 cm, a cover height of about 0.6 cm, and an overall height in the cover-closed position of about 8.6 cm. The skirt 256 of the liquid dispenser 200 can exhibit a height of about 0.17 cm.

In another aspect, the liquid dispensers 100 and 200 can exhibit, in the cover-closed position, an overall height in the range of about 7 cm to about 12 cm and an overall width in the range of about 0.8 cm to about 1.2 cm. In yet another aspect, the liquid dispensers 100 and 200 can exhibit a width of less than about 1.5 cm. In another aspect, the liquid dispenser can exhibit a width to height ratio of greater than about 1:8. In yet another aspect, the covers 108 and 208 exhibit a maximum depth about equal to the maximum depth of the container bodies 102 and 202, respectively. In still another aspect, the covers 108 and 208 exhibit a maximum width about equal to the maximum width of the container bodies 102 and 202, respectively. In still another aspect, the front walls 134 and 234 of the container bodies 102 and 202 are parallel to the rear walls 140 and 240. In yet another aspect, indicia can be applied to the container bodies 102 and 202 in the form of lettering, images or combinations thereof. In another aspect, a majority by weight of the liquid dispensers 100 and 200 comprises a metal. In still another aspect, the container bodies 102 and 202 can be unitary pieces.

Other embodiments of the present disclosure will be apparent to those skilled in the art from their consideration of the specification and practice of the present disclosure disclosed in this document. The applicant intends that the specification and examples be considered as exemplary only, with the true scope and spirit of the present disclosure being indicated by the following claims.

1. A liquid dispenser comprising:
a container body defining a container chamber therein, wherein the container body comprises a dispenser port in fluid communication with the container chamber, wherein the container body comprises a fill port in fluid communication with the container chamber;
an atomizer operably connected to the container body, wherein the atomizer is in fluid communication with the dispenser port and the container chamber;
a fill port closure operably connectable to the container body, wherein the fill port closure is movable from a fill-port-closure-open position opening the fill port to a fill-port-closure-closed position closing the fill port; and,
a cover movably connectable with the container body, wherein the cover is movable from a cover-closed. position enclosing the fill port and a cover-open position exposing the fill port, wherein the fill port closure is enclosed by the cover and the container body in the fill-port-closure-closed position and the cover-closed position.

2. The liquid dispenser of claim 1, wherein the container body comprises a first merlon and a second merlon separated from the first merlon by a crenel, wherein the first merlon comprises a first merlon chamber, wherein the second merlon comprises a second merlon chamber, and wherein the container chamber comprises the first merlon chamber and the second merlon chamber.

3. The liquid dispenser of claim 1, further comprising a hinge connecting the cover to the container body.

4. The liquid dispenser of claim 3, wherein the cover is pivotally movable on the hinge from the cover-closed-position to the cover-open position.

5. The liquid dispenser of claim 1, further comprising a magnet connecting the cover to the container body in the cover-closed position.

6. The liquid dispenser of claim 5, wherein the magnet is attached to the cover.

7. The liquid dispenser of claim 1, wherein the fill port closure comprises a thread, and wherein the fill port closure is threadably engaged to the fill port in the fill-port closure-closed position.

8. The liquid dispenser of claim 1, wherein the fill port closure comprises a resilient body, and wherein the resilient body is disposed in the fill port in the fill-port-closure-closed position.

9. The liquid dispenser of claim 1, wherein the fill port is aligned in the bottom wall of the container body.

10. The liquid dispenser of claim 9, wherein the cover encloses the bottom wall of the container body in the cover-closed-position.

11. A liquid dispenser comprising:
a container body defining a container chamber therein, wherein the container body comprises a dispenser port in fluid communication with the container chamber, wherein the container body comprises a fill port in fluid communication with the container chamber, wherein the dispenser port is formed within a first wall of the container body and the fill port is formed within a second wall of the dispenser body;
an atomizer operably connected to the dispenser port, wherein the atomizer is in fluid communication with the container chamber;
a fill port closure operably connectable to the container body, wherein the fill port closure is realignable between a fill-port-closure closed position wherein the fill port is closed and a fill-port-closure-open position wherein the fill port is open; and,
a cover movably connectable with the container body, wherein the cover is realignable between a cover-closed-position wherein the fill port is enclosed and a cover-open position wherein the fill port is exposed.

12. The liquid dispenser of claim 11, wherein the cover is hingedly mounted to the container body.

13. The liquid dispenser of claim 11, wherein the container body further comprises a first merlon and a second merlon separated from the first merlon by a crenel, wherein the first merlon comprises a first merlon top wall and the second merlon comprises a second merlon top wall, wherein the crenel comprises a crenel top wall, and wherein the dispenser port is aligned in the crenel top wall.

14. The liquid dispenser of claim 1, wherein the cover is detachable from the container body in the cover-open position.

15. The liquid dispenser of claim 14, wherein the cover comprises a magnet, and wherein the magnet connects the cover to the container body in the cover-closed position.

16. The liquid dispenser of claim 11, wherein the fill port closure comprises a thread formed thereon, and wherein the fill port closure is threadably connected to the fill port in the fill-port-closure-closed position.

17. The liquid dispenser of claim 11, wherein the fill port closure comprises a resilient body, and wherein the resilient body frictionally engages the fill port in the fill-port-closure-closed position.

18. The liquid dispenser of claim 11, wherein the second wall is the bottom wall of the container body.

19. The liquid dispenser of claim 18, wherein the container body further comprises a skirt depending from the bottom wall.

20. A liquid dispenser comprising:
a container body defining a container chamber, wherein the container body comprises the top wall and a bottom wall opposing the top wall, wherein the container body comprises a dispenser port disposed in the top wall, wherein the dispenser port is in fluid communication with the container chamber, wherein the container body comprises a fill port disposed in the bottom wall, wherein the fill port is in fluid communication with the container chamber, and wherein the container body comprises a skirt depending from the bottom wall;
a fill port closure operably connectable to the fill port, wherein the fill port closure is movable between a fill-port-closure-open position opening the fill port and a fill-port-closure-closed position closing the fill port; and,
a cover magnetically connectable to the container body, wherein the cover comprises a magnet, wherein the cover is movable between a cover-open position exposing the fill port and a cover-closed position covering the fill port, and wherein the magnet magnetically engages the container body in the cover-closed position.
